(19) United States
(12) Patent Application Publication
(10) Pub. No.: US 2016/0289569 A1
(43) Pub. Date: Oct. 6, 2016

(54) PROCESSES FOR PRODUCING AROMATIC HYDROCARBONS FROM A PYROLYSIS OIL

(71) Applicant: UOP LLC, Des Plaines, IL (US)

(72) Inventors: Lance A. Baird, Prospect Heights, IL (US); Trung Pham, Mount Prospect, IL (US)

(21) Appl. No.: 14/674,191

(22) Filed: Mar. 31, 2015

(51) Int. Cl. C10G 3/00; C10B 53/02; C10G 35/04; C07C 5/27; C07C 6/06

(52) U.S. Cl. CPC C10G 3/50; C07C 5/2729; C07C 6/06; C10G 35/04; C10B 53/02

(57) ABSTRACT

Processes for the production of aromatics from a pyrolysis oil. The pyrolysis oil may be obtained from a biomass by fast pyrolysis and may be filtered and upgraded to provide an aromatic rich hydrocarbon product. The aromatic rich hydrocarbon product may be passed to a separation zone to separate non-aromatic hydrocarbons from the aromatics. The remaining aromatics may be separated into aromatic product rich streams based upon the desired aromatics. Isomerization and transalkylation zones may be included to increase the yield depending on the product or desired aromatic. The non-aromatic hydrocarbons may be passed to a reformer to be converted to aromatics and hydrogen.

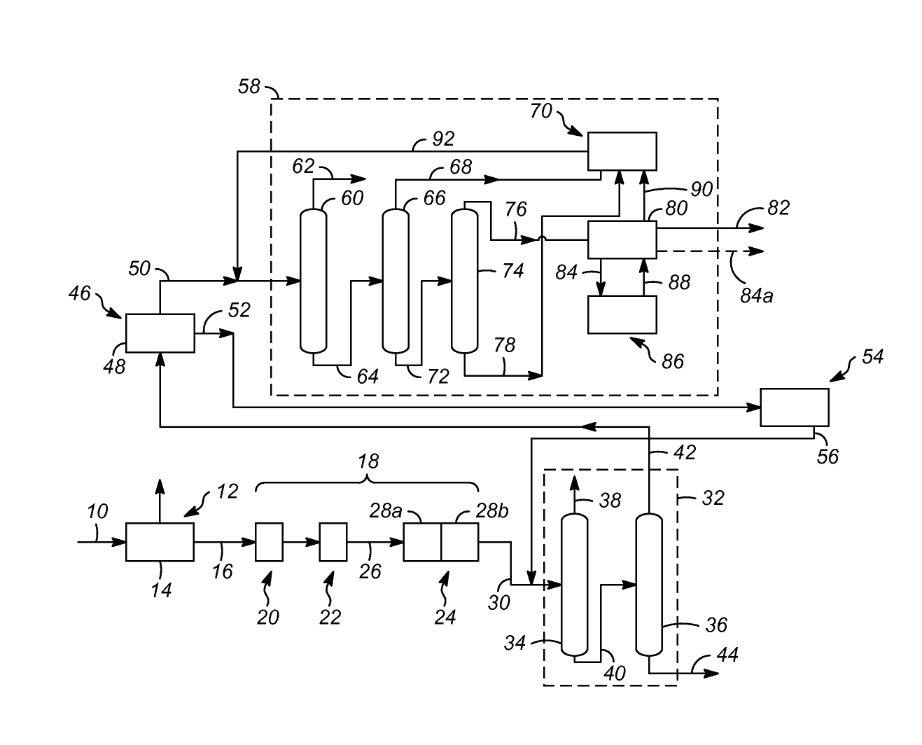

### FIELD OF THE INVENTION

This invention relates generally to processes for producing aromatic hydrocarbons to be used, for example, for the manufacture of polyethylene terephthalate, and more particularly to such processes wherein the aromatics are produced from pyrolysis of a biomass.

### BACKGROUND OF THE INVENTION

Plastics are currently manufactured primarily from fossil fuel feedstocks. One particular plastic that is desirable is polyethylene terephthalate (PET). PET resins have desirable properties for high volume drink bottling and automotive applications. In conventional processes, PET resins are manufactured from a fossil fuel source, such as petroleum crude oil, by first distilling a naphtha fraction, reforming it to a highly aromatic fraction, extracting the aromatics portion typically using a solvent such as sulfolane, then distilling the aromatics into benzene, toluene, xylene, and heavy aromatics fractions, and finally separating the paraxylene from the orthoxylene and metaxylene by adsorptive separation or by a crystalization process. The overall yield of paraxylene is commonly increased by adding transalkylation and isomerization processes to the flow scheme.

While these processes are effective for their intended purposes; however, there is growing demand for plastics derived from renewable feedstocks.

Therefore, it would be desirable to provide one or more processes for the production of aromatic hydrocarbons that may be used to produce plastics such as PET and wherein the aromatics are produced form a renewable feedstock.

### SUMMARY OF THE INVENTION

One or more processes have been invented in which an aromatic product stream, for example a paraxylene product stream, is produced from a renewable resource.

Accordingly, in a first aspect of the present invention, the present invention may be generally characterized as providing a process for producing an aromatic hydrocarbon stream from a biomass by: pyrolyzing a biomass in a pyrolysis zone to provide a pyrolysis oil; upgrading the pyrolysis oil in a conditioning zone to provide an upgraded pyrolysis oil; deoxygenating the upgraded pyrolysis oil in a deoxygenation zone having a deoxygenation catalyst to provide a deoxygenated effluent; separating a hydrocarbon stream from the deoxygenated effluent, wherein the hydrocarbon effluent stream includes non-aromatic hydrocarbons and aromatic hydrocarbons; separating the aromatic hydrocarbons of the hydrocarbon effluent stream from the non-aromatic hydrocarbons of the hydrocarbon effluent stream to provide an aromatic stream; and, separating at least one product stream from the aromatic stream.

In one or more embodiments of the present invention, the deoxygenation zone is configured to minimize any loss of aromatic hydrocarbons.

In some embodiments of the present invention, the deoxygenation zone comprises at least two reactors each comprising a deoxygenation catalyst. It is contemplated that the deoxygenation catalyst in at least one of the reactors of the deoxygenation zone includes a noble metal. It is further contemplated that the at least one of the reactors in the deoxygenation zone is operated at a temperature above 300° C. (572° F.) and below a pressure of 4100 kPa (gauge) (594.7 psig).

In at least one embodiment of the present invention, the process may include isomerizing at least a portion of the aromatic stream in an isomerization zone.

In various embodiments of the present invention, the process may include trans-alkylating at least a portion of the aromatic stream in an alkylation zone.

In one or more embodiments of the present invention, the process may include reforming the non-aromatic hydrocarbons of the hydrocarbon effluent stream in a reforming zone to provide a reformate, and, combining the reformate with the hydrocarbon effluent stream.

In at least one embodiment of the present invention, the at least one product stream comprises a paraxylene stream.

In a second aspect of the present invention, the present invention may be generally characterized as providing a process for producing an aromatic product stream from a biomass by: pyrolyzing a biomass in a pyrolysis zone to provide a pyrolysis oil stream; passing the pyrolysis oil stream to a conditioning zone configured to upgrade the pyrolysis oil stream by reducing at least one contaminant and provide an upgraded pyrolysis oil stream; passing the upgraded pyrolysis oil stream to a deoxygenation zone having a deoxygenation catalyst and being configured to provide a deoxygenated effluent stream including non-aromatic hydrocarbons and aromatic hydrocarbons; passing the deoxygenated effluent stream to a first separation zone to separate a stream having a boiling point between 80 and 200° C. (176 to 392° F.) from the deoxygenated effluent stream; passing the stream having a boiling point between 80 and 200° C. (176 to 392° F.) to a second separation zone to separate an aromatic steam and a non-aromatic hydrocarbon stream; and, passing the aromatic steam to a third separation zone configured to provide at least one product stream from the aromatic stream.

In one or more embodiments of the present invention, the second separation zone comprises an absorptive separation unit and wherein the non-aromatic hydrocarbon stream comprises a raffinate stream. It is contemplated that the process includes passing the raffinate stream to a reforming zone to provide a reformate stream, the reformate stream having a higher concentration of aromatic hydrocarbons compared to the raffinate stream, and passing the reformate stream to the first separation zone.

In various embodiments of the present invention, the deoxygenation zone comprises at least two reactors each comprising a deoxygenation catalyst. In is contemplated that the deoxygenation catalyst in at least the second reactor of the deoxygenation zone includes a noble metal. It is also contemplated that the second reactor in the deoxygenation zone is operated at a temperature above 300° C. (572° F.) and below a pressure of 4100 kPa(g) (594.7 psig).

In some embodiments of the present invention, the third separation zone includes one or more zones configured to increase an amount of desired aromatics in the at least one product stream. It is contemplated that the third separation zone includes an isomerization zone. It is further contemplated that the third separation zone includes an alkylation zone. It is also contemplated that the at least one product stream comprises a paraxylene stream. It is further contemplated that the third separation zone includes a crystallizer.

Additional embodiments, aspects, and details of the invention, which may be combined in any manner, are set forth in the following detailed description of the invention.

### DETAILED DESCRIPTION OF THE DRAWINGS

One or more exemplary embodiments of the present invention will be described below in conjunction with the following drawing FIGURE, in which:

the FIGURE shows a process flow diagram according to one or more embodiments of the present invention.

### DETAILED DESCRIPTION OF THE INVENTION

As mentioned above, one or more processes for producing aromatic compounds from the pyrolysis of a biomass have been invented. Biomass is a renewable resource and a hydrocarbon-containing oil, known as pyrolysis oil, can be produced from biomass by rapidly heating biomass to about 450 to about 600° C. (about 842 to about 1112° F.) in the absence of air using a circulating heat transfer media, such as sand. Under these conditions, a pyrolysis vapor stream including organic vapors, water vapor, and pyrolysis gases is produced, along with char (which includes ash and combustible hydrocarbon solids). The pyrolysis vapor may be condensed to produce a pyrolysis oil stream, which is a complex organic liquid that typically contains about 40 to 50% hydrocarbon, 20 to 40% oxygen and 20 to 30% by weight water. The pyrolysis oil is also rich in precursors to aromatic compounds-containing as much as 85% aromatic compounds after deoxygenation, making it very suitable as a renewable source of aromatic feedstock for various processes, such as PET resin manufacture. Accordingly, one or more processes have been invented in which an aromatic stream is produced from the pyrolysis oil.

With these general principles in mind, one or more embodiments of the present invention will be described with the understanding that the following description is not intended to be limiting.

With reference to the FIGURE, a renewable feedstock 10 is passed to a pyrolysis zone 12 and subjected to pyrolysis. The renewable feedstock 10 may comprise solid carbonaceous biomass feedstock, i.e., “biomass”, such as wood waste, agricultural waste, etc. As used in the present disclosure, the terms “renewably-based” or “renewable” denote that the carbon content of the renewable hydrocarbon (paraffins, olefins, aromatics, alkylbenzene, linear alkylbenzene or subsequent products prepared from renewable hydrocarbons), is from a “new carbon” source as measured by ASTM test method D6866-05, “Determining the Bio-based Content of Natural Range Materials Using Radiocarbon and Isotope Ratio Mass Spectrometry Analysis”, hereby incorporated by reference in its entirety. This test method measures the ¹⁴C/¹²C isotope ratio in a sample and compares it to the ¹⁴C/¹²C isotope ratio in a standard 100 mass % bio-based material to give percent bio-based content of the sample. Additionally, “bio-based materials” are organic materials in which the carbon comes from recently, on a human time scale, fixated carbon dioxide present in the atmosphere using sunlight energy, photosynthesis. On land, this carbon dioxide is captured or fixated by plant life such as agricultural crops or forestry materials. In the oceans, the carbon dioxide is captured or fixated by photosynthesizing bacteria or phytoplankton. For example, a bio-based material has a ¹⁴C/¹²C isotope ratio greater than 0. Contrarily, a fossil-based material has a ¹⁴C/¹²C isotope ratio of about 0. The term “renewable” with regard to compounds such as hydrocarbons (paraffins, olefins, di-olefins, aromatics, alkylbenzene, linear alkylbenzene etc.) also refers to compounds prepared from biomass using thermochemical methods such as (e.g., Fischer-Tropsch catalysts), biocatalysts (e.g., fermentation), or other processes, for example.

A small amount of the carbon atoms in the atmospheric carbon dioxide is the radioactive isotope ¹⁴C. This ¹⁴C carbon dioxide is created when atmospheric nitrogen is struck by a cosmic ray generated neutron, causing the nitrogen to lose a proton and form ¹⁴C, which is then immediately oxidized, to carbon dioxide. A small but measurable fraction of atmospheric carbon is present in the form of ¹⁴C. Atmospheric carbon dioxide is processed by green plants to make organic molecules during the process known as photosynthesis. Virtually all forms of life on Earth depend on this green plant production of organic molecules to produce the chemical energy that facilitates growth and reproduction. Therefore, the ¹⁴C that forms in the atmosphere eventually becomes part of all life forms and their biological products, enriching biomass and organisms which feed on biomass with ¹⁴C. In contrast, carbon from fossil fuels does not have the signature ¹⁴C/¹²C ratio of renewable organic molecules derived from atmospheric carbon dioxide. Furthermore, renewable organic molecules that biodegrade to carbon dioxide do not contribute to an increase in atmospheric greenhouse gases as there is no net increase of carbon emitted to the atmosphere. Assessment of the renewably based carbon content of a material can be performed through standard test methods such as using radiocarbon and isotope ratio mass spectrometry analysis. ASTM International (formally known as the American Society for Testing and Materials) has established a standard method for assessing the bio-based content of materials. The ASTM method is designated ASTM-D6866. The application of ASTM-D6866 to derive “bio-based content” is built on the same concepts as radiocarbon dating, but without use of the age equations. The analysis is performed by deriving a ratio of the amount of radiocarbon (¹⁴C) in an unknown sample compared to that of a modern reference standard. This ratio is reported as a percentage with the units “pMC” (percent modern carbon). If the material being analyzed is a mixture of present day radiocarbon and fossil carbon, which contains very low levels of radiocarbon, then the pMC value obtained correlates directly to the amount of biomass material present in the sample.

Returning to the Figure, in the pyrolysis zone 12 the renewable feedstock 10 is preferably subjected to fast pyrolysis. Fast pyrolysis is a thermal process during which the biomass is rapidly heated to pyrolysis temperatures of about 450 to about 600° C. (about 842 to about 1,112° F.) in the absence of air using a pyrolysis reactor 14 having a circulating heat transfer media, such as sand. Under these conditions, a pyrolysis vapor stream including organic vapors, water vapor, and pyrolysis gases is produced, along with char (which includes ash and combustible hydrocarbon solids). The pyrolysis vapor may be condensed to produce a pyrolysis oil. Biomass-derived pyrolysis oil is available from, for example, Ensyn Technologies Inc., of Ontario, Canada. The composition of biomass-derived pyrolysis oil is somewhat dependent on feedstock and processing variables. The biomass-derived pyrolysis oil may be produced, for example, from fast pyrolysis of wood biomass in a pyrolysis reactor. However, virtually any form of biomass can be considered for pyrolysis to produce biomass-derived pyrolysis oil. In addition to wood, biomass-derived pyrolysis oil may be derived from biomass material such as bark, agricultural wastes/residues, nuts and seeds, algae, grasses, forestry residues, cellulose and lignin, or the like. The biomass-derived pyrolysis oil may also be obtained by different modes of pyrolysis, such as fast pyrolysis, vacuum pyrolysis, catalytic pyrolysis, and slow pyrolysis (also known as carbonization) or the like.

As shown in the FIGURE, a pyrolysis oil stream 16 may be passed from the pyrolysis zone 12 to an upgrading zone 18 having one or more contaminant removal zones 20, 22 and a hydroprocessing zone 24. The contaminant removal zone may comprise a filtration zone 20 configured to remove particulates or other materials from the pyrolysis oil. Additionally, the contaminant removal zone may comprise an ion exchange zone 22 to remove metals from the pyrolysis oil. From the contaminant removal zones 20, 22 a purified pyrolysis oil stream 26 is passed to the hydroprocessing zone 24.

In the hydroprocessing zone 24, the pyrolysis oil in the purified pyrolysis oil stream 26 may be upgraded by removing the water and oxygen by contacting the pyrolysis oil with hydrogen and catalyst at suitable hydroprocessing conditions (i.e., deoxygenation, decarbonylation, decarboxylation) in one or more reactors. Depending on the desired length of hydrocarbons, deoxygenation via hydrodeoxygenation may be preferred over decarbonylation and decarboxylation (in which a carbon molecule is rejected in form of CO or CO₂). Suitable conditions are disclosed in, for example, U.S. application Ser. Nos. 14/551,797 and 14/101,842 filed Nov. 24, 2014 and Dec. 10, 2013, respectively, and both of which are incorporated herein by reference. It is preferred that the hydroprocessing zone 24 is configured to maximize aromatic production, for example, by using two or more reactors 28a, 28b.

In a preferred embodiment, the first reactor 28a in the hydroprocessing zone 24 may include conditions include a reaction temperature of from about 100 to about 400° C. (212 to 752° F.), or from about 150 to about 350° C. (302 to 662° F.) in different embodiments. The reaction pressure may be from about 2,000 to about 20,000 kPa (290 to 2,900 psi), or from about 3,400 to about 17,000 kPa (493 to 2,466 psi) in various embodiments. In this description, indicated pressures are gauge as opposed to absolute unless otherwise indicated. The LHSV is determined on a basis of weight of the first reactor feedstock/weight of deoxygenation catalyst/hour. The LHSV may be from about 0.10 to about 2 hr⁻¹, and the ratio of hydrogen flow to pyrolysis oil flow may be from about 1,000 to about 15,000 standard cubic feet of hydrogen per barrel of pyrolysis oil (SCF/B) and may be different between the reactors 28a, 28b in the hydroprocessing zone 24.

The second reactor 28b in the hydroprocessing zone 24 may operate with conditions for the simultaneous deoxygenation and dehydrogenation with the preservation of the aromatic compounds and include a temperature of from about 300 to about 540° C. (572 to 1,004° F.), or from about 400 to about 520° C. (752 to 968° F.), or from about 475 to about 520° C. (887 to 968° F.) in various embodiments. The pressure may be from about 340 to about 5,500 kPa (49 to 798 psi), or from about 1,000 to about 3,100 kPa (145 to 450 psi) in various embodiments. The LHSV may be from about 0.1 to about 3 hr⁻¹. Although not shown, it is contemplated that a separation zone is disposed between the reactors 28a, 28b in hydroprocessing zone 24.

Preferably, the second reactor 28b includes a higher temperature and lower pressure to shift the equilibrium and produce more aromatic hydrocarbons. Additionally, while any variety of acceptable catalysts may be used, it is preferred that the catalyst in at least the second reactor 28b of the hydroprocessing zone 24 comprises a catalyst having one or more noble metals.

From the hydroprocessing zone 24, a deoxygenated effluent stream 30 comprising non-aromatic hydrocarbons (including, linear, branched, olefins, and cyclic hydrocarbons) and aromatic hydrocarbons is passed to a first separation zone 32. In the depicted embodiment, which is not intended to be limiting, the first separation zone comprises two columns 34, 36. In the first column 34, a light ends stream 38 comprising C₅₋ hydrocarbons will be separated from the deoxygenated effluent stream 30. A bottoms stream 40 from the first column 34 may be passed to the second column 36 to separate a hydrocarbon effluent stream 42 comprising a mixture of the C₆ to C₁₀ aromatic hydrocarbons and some non-aromatic hydrocarbons having similar boiling points. In a preferred embodiment the hydrocarbon effluent stream 42 comprises non-aromatic hydrocarbons and aromatic compounds that have a boiling point between 80 and 200° C. (176 to 392° F.). A heavy ends stream 44 comprising C₁₁₊ hydrocarbons can be withdrawn from the bottoms of the second column 36. The further processing of the light ends stream 38 and the heavy ends stream 44 are not necessary for a practicing of the present invention.

In at least one embodiment of the present invention, the hydrocarbon effluent stream 42 comprising non-aromatic hydrocarbons and aromatic compounds that have a boiling point between 80 and 200° C. (176 to 392° F.) is passed from the first separation zone 32 to a second separation zone 46. In the second separation zone 46, the non-aromatic hydrocarbons may be separated from the aromatic hydrocarbons with, for example, an absorptive separation unit 48. The absorptive separation unit 48 comprises one or more vessels and utilizes a liquid solvent to extract the non-aromatic hydrocarbons into an extract stream 50 comprising aromatic hydrocarbons and a raffinate stream 52 comprising the non-aromatic hydrocarbons. The operation of such an absorptive separation unit 48 is known in the art.

It is contemplated that the raffinate stream 52 is passed to a reforming zone 54 in which the non-aromatic hydrocarbons may be reformed into aromatic hydrocarbons. An effluent 56 from the reforming zone 54, or a reformate, may be passed to the first separation zone 32 in order to separate out the components as discussed above.

Reforming is a catalytic process for producing aromatics from paraffins and naphthenes by rearranging or restructuring hydrocarbon molecules and breaking larger hydrocarbon molecules into smaller ones. Hydrogen is produced as a byproduct. An example reforming process is the CCR PLATFORMING™ catalytic reforming process (UOP, Des Plaines, Ill.), which includes dehydrogenation of naphthenes, isomerization of paraffins and naphthenes, dehydrogenation of paraffins, paraffin hydrocracking, and dealkylation of aromatics. An example reforming process includes a catalyst that is continuously regenerated in a regeneration section. Example conditions include a pressure between about 344 and about 1,379 kPa (50 to 200 psig), a temperature of between about 510 and about 566 ° C. (950 to 1,050° F.). Example catalysts include platinum and tin catalysts. Liquid hourly space velocity (LHSV) can vary, and can be selected to provide a desired reformate such as the effluent 56.

Returning to the extract stream 50 which comprises an aromatic hydrocarbon stream, the various aromatic compounds can be separated out into one or more product streams in a third separation zone 58. A preferred separation zone 58 is shown, in which the aromatic stream 50 is passed to a first fractionation column 60 configured to provide a benzene rich stream 62 which may be recovered as a product stream or processed further depending on the requirements of the processor. A bottoms stream 64 from the first fractionation column 60 may be passed to a second fractionation column 66.

The second fractionation column 66 is configured to separate a toluene rich stream 68. The toluene rich stream 68 may be passed to a transalkylation zone 70 (which is discussed in more detail below). A bottoms stream 72 from the second fractionation column 66 may be passed to a third fractionation column 74.

The third fractionation column 74 is configured to separate a xylene rich stream 76, which also may comprise ethylbenzene, from a bottoms stream 78 which may be passed to the transalkylation zone 70. The xylene rich stream 76 may be passed to a xylene separation unit 80, which may comprise a crystallizer, an adsorptive separation unit, or both. In the xylene separation unit 80, a desired product stream 82, such as a paraxylene stream, may be provided. A metaxylene stream (not shown), may also be provided using an adsorptive separation (MX Sorbex®) in combinations with other units in the xylene separation unit 80. As mentioned at the outset, the paraxylene, derived from a renewable feedstock, can be used to make plastics, such as PET.

In one embodiment in which the desired product stream 82 comprises a paraxylene stream, the xylene separation unit 80 may also provide a orthoxylene, metaxylene, and ethylbenzene stream 84, which may be passed to an isomerization zone 86 to convert some of the orthoxylene, metaxylene, and ethylbenzene into paraxylene, and an isomerization effluent 88 may be returned to the xylene separation unit 80. Alternatively, in embodiments without an isomerization zone 86, the orthoxylene, metaxylene, and ethylbenzene stream 84a (shown in dashed lines) may be recovered and processed further.

In some embodiments, a heavy aromatic stream 90 may be passed from the xylene separation unit 80 to the transalkylation zone 70. In the transalkylation zone 70, toluene, heavy aromatics (trimethylbenzenes, methyl-ethylbenzenes and C₁₀ aromatics), and the compounds from the bottoms stream 78 from the third fractionation column 74 may undergo various transalkylation reactions to convert some of the aromatic compounds into xylenes and benzene. Transalkylation is a chemical reaction resulting in transfer of an alkyl group from one organic compound to another. Catalysts, particularly zeolite catalysts, are often used to effect the reaction. If desired, the transalkylation catalyst may be metal stabilized using a noble metal or base metal, and may contain suitable binder or matrix material such as inorganic oxides and other suitable materials. The transalkylation catalyst is usefully disposed as a fixed bed in a reaction zone of a vertical tubular reactor, with the feed stock charged through the bed in an upflow or downflow manner. The transalkylation zone 70 normally operates at conditions including a temperature in the range of about 130 to about 540° C. (266 to 1,004° F.), or from about 200 to about 520° C. (392 to 968° F.). The transalkylation zone is typically operated at moderately elevated pressures broadly ranging from about 100 kPa to about 10 MPa (14.5 to 1450 psi) absolute, or from about 1 MPa to about 4.2 MPa (145 to 609 psi) (absolute). The transalkylation reaction can be effected over a wide range of space velocities. That is, volume of charge per volume of catalyst per hour;

weight hourly space velocity (WHSV) generally is in the range of from about 0.1 to about 30 hr⁻¹, or from about 0.5 to about 10 hr⁻¹ Hydrogen to hydrocarbon molar ratio is in the range of from about 0.5 to 15, or from about 1 to 5. The catalyst is typically selected to have relatively high stability at a high activity level.

A transalkylated effluent stream 92 from the transalkylation zone 70 comprises unconverted compounds and a product containing at least benzene and additional xylenes. The transalkylated effluent stream 92 may be combined with the extract stream 50 from the second separation zone 46 to process and separate out the aromatic compounds as discussed above.

While the foregoing description included paraxylene as the desired aromatic, the third separation zone 58 and the separation units therein can be adjusted and tailored based upon the desired aromatic compound. Accordingly, the depicted embodiment for producing the paraxylene product stream should not be taken to be limited, but only exemplary.

Nevertheless, for any desired aromatic compound, in the processes described herein, an aromatic product stream has been produced from a renewable resource. The aromatic product stream may be paraxylene which can be used in the manufacture of polyethylene terephthalate.

It should be appreciated and understood by those of ordinary skill in the art that various other components such as valves, pumps, filters, coolers, etc. were not shown in the drawings as it is believed that the specifics of same are well within the knowledge of those of ordinary skill in the art and a description of same is not necessary for practicing or understanding the embodiments of the present invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A process for producing an aromatic hydrocarbon stream from a biomass, the process comprising:
pyrolyzing a biomass in a pyrolysis zone to provide a pyrolysis oil;
upgrading the pyrolysis oil in a conditioning zone to provide an upgraded pyrolysis oil;
deoxygenating the upgraded pyrolysis oil in a deoxygenation zone having a deoxygenation catalyst to provide a deoxygenated effluent;
separating a hydrocarbon effluent stream from the deoxygenated effluent, wherein the hydrocarbon effluent stream includes non-aromatic hydrocarbons and aromatic hydrocarbons;
separating the aromatic hydrocarbons of the hydrocarbon effluent stream from the non-aromatic hydrocarbons of the hydrocarbon effluent stream to provide an aromatic stream; and,
separating at least one product stream from the aromatic stream.

2. The process of claim 1 wherein the deoxygenation zone is configured to minimize any loss of aromatic hydrocarbons.

3. The process of claim 1 wherein the deoxygenation zone comprises at least two reactors each comprising a deoxygenation catalyst.

4. The process of claim 3 wherein the deoxygenation catalyst in at least one of the reactors of the deoxygenation zone includes a noble metal.

5. The process of claim 3 wherein at least one of the reactors in the deoxygenation zone is operated at a temperature above 300° C. and below a pressure of 4100 kPa-g.

6. The process of claim 1 further comprising:
isomerizing at least a portion of the aromatic stream in an isomerization zone.

7. The process of claim 1 further comprising:
trans-alkylating at least a portion of the aromatic stream in an alkylation zone.

8. The process of claim 1 further comprising:
reforming the non-aromatic hydrocarbons of the hydrocarbon effluent stream in a reforming zone to provide a reformate; and,
combining the reformate with the hydrocarbon effluent stream.

9. The process of claim 1 wherein the at least one product stream comprises a paraxylene stream.

10. A process for producing an aromatic product stream from a biomass, the process comprising:
pyrolyzing a biomass in a pyrolysis zone to provide a pyrolysis oil stream;
passing the pyrolysis oil stream to a conditioning zone configured to upgrade the pyrolysis oil stream by reducing at least one contaminant and provide an upgraded pyrolysis oil stream;
passing the upgraded pyrolysis oil stream to a deoxygenation zone having a deoxygenation catalyst and being configured to provide a deoxygenated effluent stream including non-aromatic hydrocarbons and aromatic hydrocarbons;
passing the deoxygenated effluent stream to a first separation zone to separate a stream having a boiling point between 80 and 200° C. from the deoxygenated effluent stream;
passing the stream having a boiling point between 80 and 200° C. to a second separation zone to separate an aromatic steam and a non-aromatic hydrocarbon stream; and,
passing the aromatic steam to a third separation zone configured to provide at least one product stream from the aromatic stream.

11. The process of claim 10 wherein the second separation zone comprises an absorptive separation unit and wherein the non-aromatic hydrocarbon stream comprises a raffinate stream.

12. The process of claim 11 further comprising:
passing the raffinate stream to a reforming zone to provide a reformate stream, the reformate stream having a higher concentration of aromatic hydrocarbons compared to the raffinate stream; and,
passing the reformate stream to the first separation zone.

13. The process of claim 10 wherein the deoxygenation zone comprises at least two reactors each comprising a deoxygenation catalyst.

14. The process of claim 13 wherein the deoxygenation catalyst in at least one of the reactors of the deoxygenation zone includes a noble metal.

15. The process of claim 14 wherein the at least one of the reactors in the deoxygenation zone is operated at a temperature above 300° C. and below a pressure of 4100 kPa-g.

16. The process of claim 10 wherein the third separation zone includes one or more zones configured to increase an amount of desired aromatics in the at least one product stream.

17. The process of claim 16 wherein the third separation zone includes an isomerization zone.

18. The process of claim 17 wherein the third separation zone includes an alkylation zone.

19. The process of claim 18 wherein the at least one product stream comprises a paraxylene stream.

20. The process of claim 19 wherein third separation zone includes a crystallizer.
